(19) United States
(12) Patent Application Publication
(10) Pub. No.: US 2023/0226234 A1
(43) Pub. Date: Jul. 20, 2023

(54) DECONTAMINATION USING ULTRAVIOLET (UV) LIGHT SYSTEM AND METHOD FOR DECONTAMINATING LIQUIDS USING ULTRAVIOLET (UV) LIGHT SYSTEM IN COMBINATION WITH AN ADVANCE OXIDATION PROCESS

(71) Applicant: ECOSPEARS, INC., Altamonte Springs, FL (US)

(72) Inventors: Sergie A. Albino, Longwood, FL (US); Devon W. Miller, Altamonte Springs, FL (US)

(21) Appl. No.: 18/080,518

(22) Filed: Dec. 13, 2022

Related U.S. Application Data
(60) Provisional application No. 63/288,759, filed on Dec. 13, 2021.

(51) Int. Cl. A61L 2/10; A61L 2/08

(52) U.S. Cl. CPC A61L 2/10; A61L 2/088; A61L 2202/11

(57) ABSTRACT

A system that includes one or more quartz-sleeveless reactors to purify contaminated liquid in series or parallel. Each quartz-sleeveless reactor includes a continuous and independent reactor chamber. The system includes at least one continuous-batch flow, interior chamber reactor housed in the reactor chamber. Each interior chamber reactor of the at least one interior chamber reactor includes an ultraviolet (UV) lamp to emit UV radiation and fluid transport chamber. Each interior chamber reactor passes a stream of a mixture in the fluid transport chamber and around the UV lamp. The mixture includes an advanced oxidative process (AOP) additive and contaminated liquid. Each interior chamber reactor radiates the mixture while in the chamber with the emitted UV radiation from the UV lamp, simultaneously cools the UV lamp with the mixture, and autonomously passes a radiated resultant mixture into the reactor chamber.

### CROSS-REFERENCE TO RELATED APPLICATIONS

This application claims priority benefit of U.S. Provisional Application No. 63/288,759, titled “SYSTEM AND METHOD FOR WATER PURIFICATION USING ULTRAVIOLET (UV) LIGHT,” filed Dec. 13, 2021, which is incorporated herein in its entirety.

### BACKGROUND

Embodiments relate to a liquid decontamination and, more particularly, to a system and method for liquid decontamination using ultraviolet lights in combination with an advance oxidation process.

Water for human consumption can have contaminates that should meet certain governmental rules and regulations. For example, water can have polychlorinated biphenyls (PCBs) which can be unsafe at certain levels for human consumption and may cause cancer in certain individuals of the population. The Environmental Protection Agency (EPA) has set a PCB level in public systems of approximately 0.0005 ppm (parts per million). Although PCBs have been banned by some governments, PCBs still exist.

Water that is near landfills or industrial sites with hazardous water materials can become contaminated with PCBs. Levels of other water contaminates can vary based on geographical location. Common water contaminates include viruses, bacteria and microorganisms, for example. Some chemical compounds or chemicals naturally found in the environment can contaminate water such as aluminum (Al), fluoride, Arsenic (As) and nitrates (NO₃).

Accordingly, there remains a need to remove PCBs and other contaminates from water or liquids.

### SUMMARY

The embodiments herein relate to a system and methods for liquid decontamination.

An aspect of the embodiments includes a system including one or more main reactors, each main reactor includes at least one continuous and independent interior reactor chamber and one or more interior chamber reactors. Each interior chamber reactor includes one or more ultraviolet (UV) lamps to emit UV radiation and fluid transport chamber. Each interior chamber reactor is configured to pass a stream of a mixture in the fluid transport chamber and around the UV source, the mixture comprising an advanced oxidative process (AOP) additive and contaminated liquid; radiate the mixture while in the chamber with the emitted UV radiation from the UV source; simultaneously cool the UV source with the mixture; and autonomously pass a radiated resultant mixture into the main reactor chamber.

An aspect of the embodiments includes an interior chamber reactor that includes a cover having an electrical connector to receive power from an ultraviolet ballast and a fluid connector. The interior chamber reactor includes an ultraviolet (UV) lamp or UV source to emit UV radiation and electrically connected to the electrical connector in the cover. The interior chamber reactor includes a UV transmissive fluid transport chamber made of UV transmissive lens material. The fluid transport chamber passes a stream of a mixture through the fluid transport chamber coiled around the UV source, the mixture comprising an advanced oxidative process (AOP) additive and contaminated liquid, passes the UV radiation by the UV source(s) along a length of the chamber to radiate the mixture while in the chamber, and passes a radiated resultant mixture into a main reactor chamber such that the radiated resultant mixture continues to receive the UV radiation produced by the UV source to further treat the radiated resultant mixture and simultaneously cool the UV source by the radiated resultant mixture.

An aspect of the embodiments includes a method that includes performing an ultraviolet advanced oxidation process (AOP) on a mixture using a system with at least one UV interior chamber reactor, the mixture comprising an AOP additive and a contaminated liquid; during the AOP, performing double dosing of UV radiation on the mixture; causing photocatalysis within the mixture to decontaminate the liquid; and simultaneously, cooling UV sources of the system with the treated mixture during double dosing of the UV radiation.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 shows a perspective view of a liquid decontamination system according to an embodiment;

FIG. 2A shows components of the liquid decontamination system of FIG. 1 within the housing frame according to an embodiment;

FIG. 2B shows additional components of the liquid decontamination system of FIG. 2A according to an embodiment;

FIG. 3A shows a top view of the liquid decontamination system of FIG. 1 within the housing frame according to an embodiment;

FIG. 3B shows a top view of the liquid decontamination system of FIG. 3A with interior chamber reactor power wiring added according to an embodiment;

FIG. 4 shows a front perspective view of an ultraviolet (UV) interior chamber reactor;

FIG. 5 shows an interior view of an interior reactor chamber according to an embodiment;

FIG. 6 shows a front view of the control panel according to an embodiment;

FIG. 7A shows a perspective view of a second ultraviolet (UV) interior chamber reactor;

FIG. 7B shows a front view of the second ultraviolet (UV) interior chamber reactor of FIG. 7A;

FIG. 8A shows a side perspective view of a baffle according to an embodiment;

FIG. 8B shows a front view of a baffle of FIG. 8A according to an embodiment;

FIG. 9 shows an interior view of the reactor according to an embodiment;

FIG. 10 shows a top view of the reactor of FIG. 9;

FIG. 11 shows an interior view of a tubular reactor according to an embodiment;

FIG. 12 shows a flow diagram of a method for decontaminating a contaminated liquid;

FIG. 13 shows a front view of the third ultraviolet (UV) interior chamber reactor;

FIG. 14 shows the third ultraviolet (UV) interior chamber reactor with the outer chamber removed;

FIG. 15A shows a perspective view of components of the liquid decontamination system of FIG. 1 within the housing frame according to an embodiment;

FIG. 15B shows a top view of the liquid decontamination system of FIG. 15A within the housing frame according to an embodiment;

FIG. 16 shows an interior view of the reactor according to an embodiment; and

FIG. 17 shows a top view of the reactor of FIG. 16.

### DETAILED DESCRIPTION

Embodiments are described herein with reference to the attached figures wherein like reference numerals are used throughout the figures to designate similar or equivalent elements. The figures are not drawn to scale and they are provided merely to illustrate aspects disclosed herein. Several disclosed aspects are described below with reference to non-limiting example applications for illustration. It should be understood that numerous specific details, relationships, and methods are set forth to provide a full understanding of the embodiments disclosed herein. One having ordinary skill in the relevant art, however, will readily recognize that the disclosed embodiments can be practiced without one or more of the specific details or with other methods. In other instances, well-known structures or operations are not shown in detail to avoid obscuring aspects disclosed herein. The embodiments are not limited by the illustrated ordering of acts or events, as some acts may occur in different orders and/or concurrently with other acts or events. Furthermore, not all illustrated acts or events are required to implement a methodology in accordance with the embodiments.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

The systems and methods of the disclosure provide an ex-situ pump and treat process to destroy chlorinated contamination in groundwater or other contaminated liquids. The systems and methods of the disclosure process liquid, for example, uses an Ultraviolet Advanced Oxidation Process (UV/AOP). The UV/AOP process mixes hydrogen peroxide (H₂O₂), but not limited to, in the liquid and introduces ultraviolet light C (UV-C) inside a reactor via UV sources. This causes the hydrogen peroxide to be broken down into free radicals that target and destroy the PCBs. The system may be configured to achieve greater than 90% PCB degradation in groundwater (i.e., 90% reduction is 1 Log Reduction, 99% is 2 Log Reduction, etc.). By way of non-limiting example, UV-C is a wavelength in the range of approximately 100-280 nanometers (nm) or other germicidal light. As used herein, the term UV-C light may sometimes be referred to as a “germicidal ultraviolet light.”

The PCB degradation was determined using both the US EPA 8082 Method and the US EPA 1668 Method. The US EPA 8082 Method is a low-resolution analytical method that has a limitation to achieving really low concentration figures (i.e., limit of 80-85% effectiveness); and when the US EPA 1668 Method is used, the same figures could be above 90%. This is because the US EPA 1668 Method is able to see to the individual congeners for the calculation.

The embodiments herein are directed to a system that includes one or more main chamber reactors. Each reactor includes a main reactor chamber and at least one A system that includes one or more quartz-sleeveless reactors to purify contaminated liquid in series or parallel. Each quartz-sleeveless reactor includes a reactor chamber. The system includes at least one continuous-batch flow, interior chamber reactor housed in the reactor chamber. Each interior chamber reactor of the at least one interior chamber reactor includes an ultraviolet (UV) lamp to emit UV radiation and fluid transport chamber. Each interior chamber reactor passes a stream of a mixture in the fluid transport chamber and around the UV source. The mixture includes an advanced oxidative process (AOP) additive and contaminated liquid. Each interior chamber reactor radiates the mixture while in the chamber with the emitted UV radiation from the UV source, simultaneously cools the UV source with the mixture, and autonomously passes a radiated resultant mixture into the reactor chamber.

Each continuous-batch flow, interior chamber reactor includes at least one ultraviolet (UV) source to emit UV radiation and fluid transport chamber made of one of: UV transmissive material and UV non-transmissive material. The interior chamber reactor is configured to: pass a stream of a mixture around the UV source in the interior chamber reactor, the mixture comprising an advanced oxidative process (AOP) additive and contaminated liquid; simultaneously cool the UV source with the mixture; radiate the mixture with the UV radiation while in the fluid transport chamber; and autonomously pass a radiated resultant mixture (i.e., treated mixture) into the reactor chamber.

In some embodiments, the system includes at least one interior chamber reactor housed in the reactor chamber. Each interior chamber reactor of the at least one interior chamber reactor includes an ultraviolet (UV) lamp to emit UV radiation and fluid transport chamber made of UV transmissive lens material. Each interior chamber reactor is configured to pass a stream of a mixture through the chamber, the mixture comprising an advanced oxidative process (AOP) additive and contaminated liquid, radiate the mixture while in the chamber, simultaneously cool the UV source with the radiated mixture, and autonomously pass a radiated resultant mixture into the reactor chamber such that the radiated resultant mixture continues to receive the UV radiation produced by the UV source of any one or more interior chamber reactors of the at least one interior chamber reactor to further treat the radiated resultant mixture.

The system may employ a reactor design, where the fluid transport chamber includes UV transmissive chamber with an effluent that autonomously evacuates inside the main reactor, that incorporates a non-plug flow interior chamber reactor design to 1) increase UV dosing by 2) increasing residence time. Additionally, if advanced oxidation process (APO) are incorporated, such as using H₂O₂ or titanium dioxide (TiO₂), increased residence time also increases the chances of chemical destruction via oxidation. Hydrogen Peroxide (H₂O₂) and titanium dioxide (TiO₂) are examples of an advanced additive process (AOP) additive.

The embodiments herein have application for a variety of liquids such as Ethanol, transformer oil, mineral oil, water, groundwater, reclaimed liquid, or other fluids.

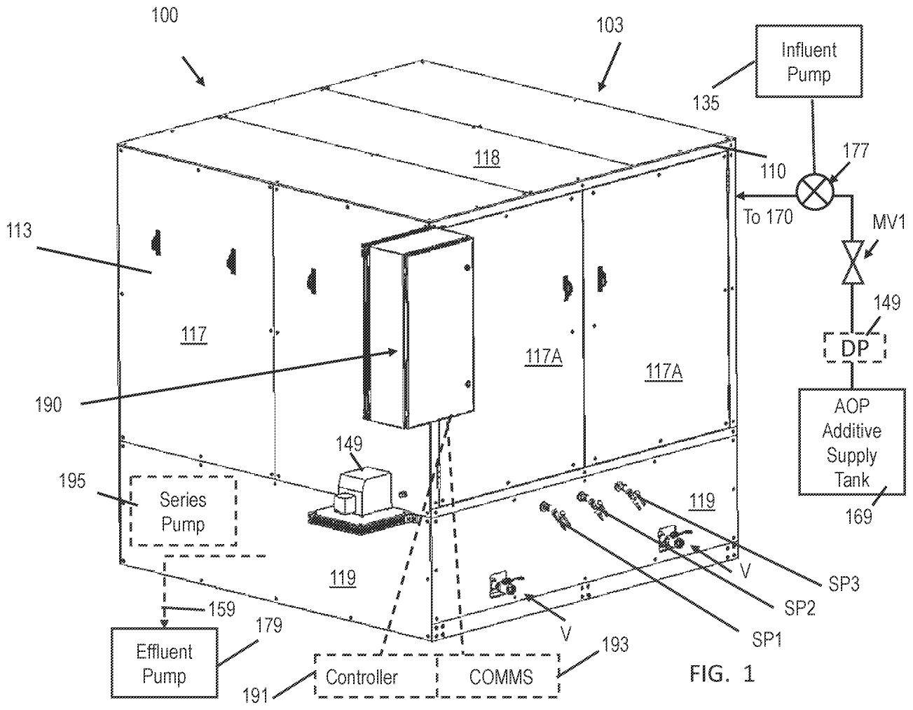

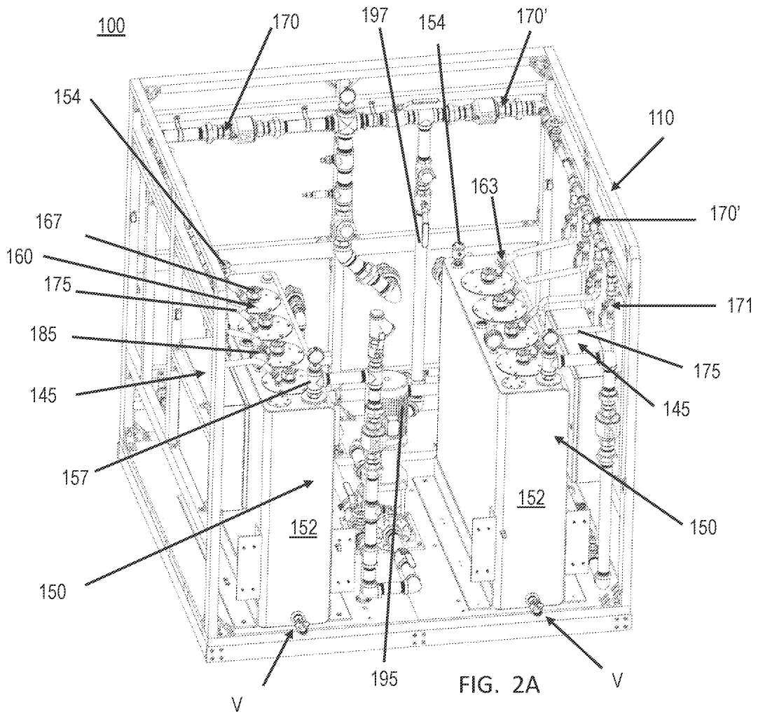

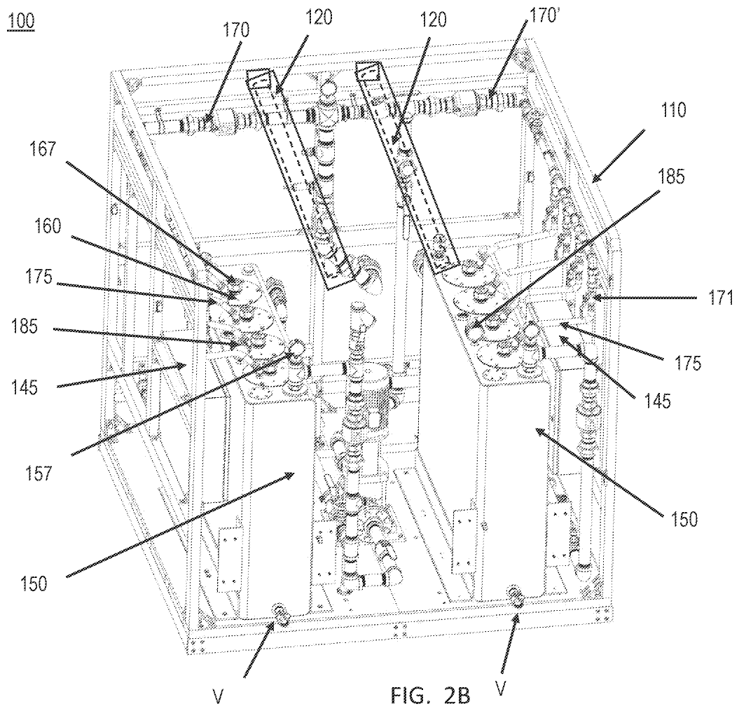

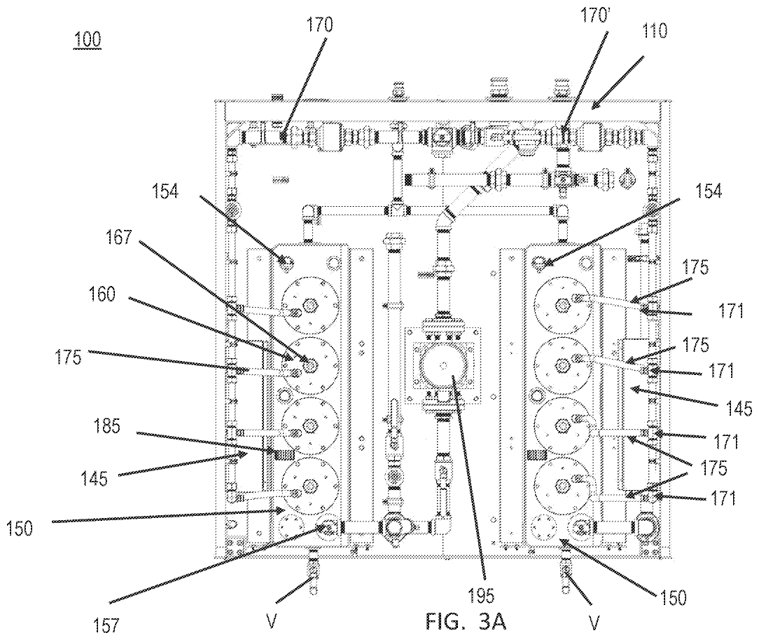

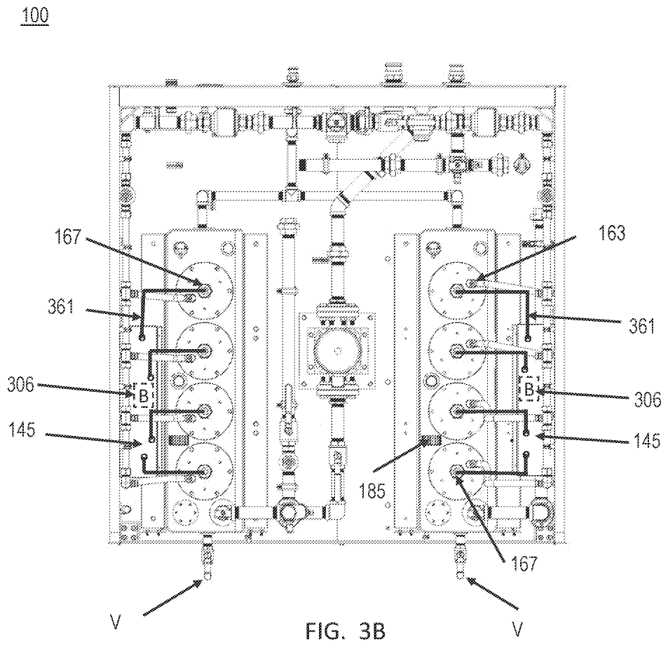

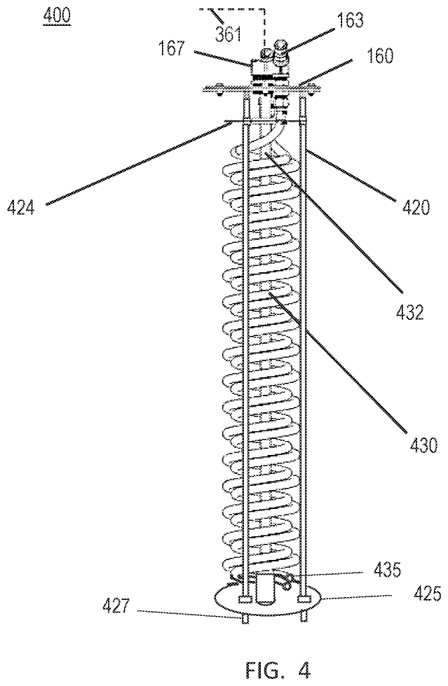

Referring to FIGS. 1, 2A, 2B, 3A and 3B, various components of the liquid decontamination system 100 are shown. FIGS. 2A, 2B, 3A and 3B show components of a liquid decontamination system 100 within a housing frame 110 according to an embodiment. FIG. 2A shows components of the liquid decontamination system of FIG. 1 within the housing frame 110 according to an embodiment. FIG. 2B shows additional components of the liquid decontamination system 100 of FIG. 2A according to an embodiment. FIG. 3A shows a top view of the liquid decontamination system 100 of FIG. 1 within the housing frame 110 according to an embodiment. FIG. 3B shows a top view of the liquid decontamination system 100 of FIG. 3A with interior chamber reactor power wiring added according to an embodiment. FIG. 4 shows a front perspective view of an ultraviolet (UV) interior chamber reactor for decontamination using an AOP process.

With reference to FIG. 1, the system 100 is housed in housing 103 that may include a frame 110 and side walls 113. One or more of the side walls may include panels 117, which may illuminate the light of one or more illuminating devices 120 (FIG. 2B). The one or more illuminating devices 120 may illuminate a different color of a plurality of colors, based on the status of the system 100. The system 100 may include an AOP additive supply tank 169 that may hold an amount of hydrogen peroxide (H₂O₂) or titanium dioxide (TiO₂) for photocatalytic oxidation using a UV source. The UV source is within the interior chamber reactor and will be described later. The AOP additive supply tank 169 may be coupled to a dosing pump (DP) 149. The dosing pump 149 may be coupled to metered valve MV1. The dosing pump 149 is shown affixed to the side of the housing 103.

The system 100 may comprise a control station 190. The control station 190 may include a controller 191 or control logic for controlling the operation of the system 100. The controller 191 may include a processor or microprocessor. The control station 190 may send control signals to activate pumps and other electrical instrumentation via wireless or wired communications protocols via communication unit (COMMS) 193. Communications protocols may include short range communication protocols, near field communication protocols, and/or long-range communication protocols. The control station 190 may receive signals via COMMS 193 from one or more electrical instruments and control the one or more illuminating devices 120 to change the color of illumination, as will be discussed later. The housing 103 may include top panels 118. The side walls 113 may include a skirt 119 below the panels 117. The skirt 119 may be opaque. Each electrical component controlled by the control station 190 also includes a communication unit (not shown) to communicate using wireless or wired communication protocols.

In some embodiments, the housing 103 is shaped into a cube, rectangle, or box. At least one side wall may include hinged doors 117A made of the illuminating panels. In some embodiments, the housing 103 can have other shapes including a tube-shaped housing. The housing may have other geometric shapes depending on the available space or shape in which the system is deployed. In some embodiments, the system 100 may include sampling ports SP1, SP2 and SP3. The first sampling port SP1 allows a technician to sample the input mixture that includes the AOP additive and contaminated liquid. The second sampling port SP2 allows a technician to sample the treated liquid in the first reactor. The third sampling port SP3 allows a technician to sample the treated mixture in the second reactor. For example, the treatment of the mixture in the first reactor may be sufficient without the need to pass the treated mixture into the second reactor via a series pump 195.

In this design, both influent and effluent pumps 135 and 179 may be outside of the housing 103. Only the series pump 195, denoted in dashed lines in FIG. 1, may be located within the housing 103. In some embodiments where the system 100 is stand-alone technology, or “flange-to-flange” design, both influent and effluent pumps 135 and 179 will be incorporated inside the housing 103. Effluent pump 179 may be coupled to an effluent line 159, denoted in dashed line, as the line would be inside of the housing 103.

Both the H₂O₂ or AOP supply tank 169 and the metering valve MV1 may be outside of the housing for easier access, in some embodiments. In other embodiments, the H202 or AOP supply tank 169 and the metering valve MV1 may be within the housing.

Referring now to FIGS. 2A, 2B, 3A, 3B and 5, the system 100 includes one or more reactors 150. In the illustration, two reactors 150 are shown. However, the system 100 may be sized to accommodate an amount of liquid that needs decontamination. Each reactor 150 includes a reactor chamber 152 with a plurality of orifices for loading the ultraviolet (UV) interior chamber reactor 400 (FIG. 4); only the interior chamber reactor cover 160 is shown in FIGS. 2A, 2B, 3A, 3B and 5. Each reactor chamber 152 is a continuous and/or independent interior reactor chamber to perform decontamination therein.

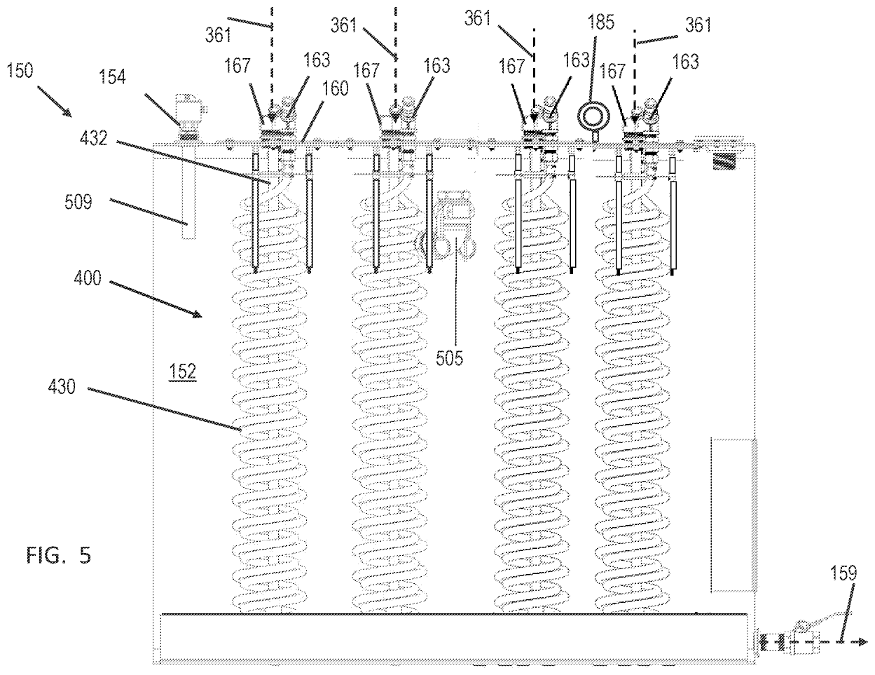

The reactor 150 may have a first gauge 154 connected to the reactor chamber 152. The first gauge 154 measures effluent pipping pressure. The gauge 154 is in communication with controller 191 (FIG. 1) to control the pumps in the system 100. The reactor 150 may have a second gauge 185 connected to the reactor chamber 152. The reactor chamber 152 includes chamber walls that form an enclosure with orifices and other ports or outlets. The second gauge 185 measures the reactor pressure. The reactor chamber 152 may include various ports with valves V connected thereto. For the sake of brevity, only some of the values and ports are labeled. The reactors may have an internal baffle reactor design, as will be described later. The reactor 150 may include a light sensor 505 (FIG. 5) such as, without limitation, a UV light sensor 505.

The system 100 may include fluid pipes 170 which allow certain liquids to flow to the liquid interfaces 175, each liquid interface 175 being connected to cover 160 of the UV interior chamber reactors (FIG. 4) via a liquid connector 163. Fluid pipes 170 may be influent lines that are connected to an influent pump 135. For example, pipes 170 may transport the contaminated liquid, such as liquid to the reactor 150. The fluid pipes 170 may include pipe branches 171. Each pipe branch 171 is connected to a liquid interface 175. The liquid interface 175 may be fluidly coupled via liquid connector 163 to the UV interior chamber reactor. A liquid interface 175 may include flexible chamber.

Assume that the reactor on the left side of the page is a first reactor. The first reactor includes an outlet line 157 that is coupled to the series pump 195, as seen in FIG. 2A. The treated mixture within the first reactor may exit the reactor chamber 152 via the valve V. Alternately, if the treated mixture needs further processing, the treated mixture in the first reactor is treated again by a second reactor (the reactor on the right side of the page). The treated mixture from the first reactor leaves the reactor chamber through the outlet line 157 to series pump 195 and through a return line 197. The return line 197 connects to the fluid pipes 170′ which allow certain liquids to flow to the liquid interfaces 175 of the second reactor, each liquid interface 175 being connected to cover 160 of the UV interior chamber reactors 400 (FIG. 4) via a liquid connector 163. Fluid lines 170 flow to the first reactor while the fluid lines 170′ flow to the second reactor.

In some embodiments the untreated mixture may be fed to both the first reactor and the second reactor using fluid lines 170 and 170′.

The system 100 may include one or more chambers 145 to house a source of hydrogen peroxide or other AOP additive such as AOP additive supply tank 169. The one or more chambers 145 may house the UV ballast (B) 306 that power the UV sources. The cover 160 of the UV interior chamber reactor 400 includes an electrical connector 167. As best seen in FIG. 3B, flexible chamber/cable 361 is provided to transfer the power to the UV interior chamber reactor 400 to power the UV source (FIG. 4). The UV interior chamber reactor 400 includes fasteners to fasten the cover 160 to the reactor chamber 152.

FIG. 4 shows a perspective view of an ultraviolet (UV) interior chamber reactor 400. The UV interior chamber reactor 400 has a continuous-batch flow, interior chamber reactor design, meaning that the interior chamber reactor and the fluid transport chamber 430 do not have a plug to prevent the autonomous flow of egress of the mixture. In FIG. 4, the fluid transport chamber 430 is made of UV transmissive material. FIG. 5 shows an interior view of the reactor 150 according to an embodiment. In this embodiments, there are four UV interior chamber reactors 400. However, the reactor 150 is not limited in the number of UV interior chamber reactors 400. The reactor 150 may be a quartz-sleeveless reactor 150 such that it does not include a quartz sleeve. The interior chamber reactor cover 160 allows the interior chamber reactor 400 to be removed and replaced from the reaction chamber. The UV source is also removable and replaceable from the interior chamber reactor. It should be understood from the description herein that the continuous and independent interior reactor chamber 152 allows for an independent second dosing of UV radiation independent from the UV radiation dosing by the interior chamber reactors 400. The second dosing of UV radiation may be provided by the UV lamps of the interior chamber reactors 400 or independent UV light emitting diodes (LEDs) on the interior walls of chamber 152.

The interior chamber reactor 400 includes an upper substrate 424 below the cover 160 and a bottom substrate 425. The upper and bottom substrates 424 and 425 have coupled thereto support rods 420. The support rods 420 may terminate below the bottom substrate 425 and includes support feet 427. The rods 420 extend through the upper substrate 424 and may also connect to the cover 160. Additional substrates may be provided to add support to the interior chamber reactor 400. The UV source is supported in a center of the substrates 424 and 425. The substrates 424 and 425 have a diameter which is smaller than the diameter of the cover 160 so that the substrates can be received through the orifices.

To prevent overcrowding in FIG. 5, portions of the rods 420 have been removed.

Referring still to FIG. 4, the UV interior chamber reactor 400 also includes a UV source having a transmissive light tube 432. In this embodiment, the UV transmissive, light tube 432 is a center-mounted tube. The center-mounted tube is a UV source. Hereinafter, the terms UV transmissive tube and UV source may both use the reference numeral 432. The UV interior chamber reactor also may include a fluid transport chamber 430 where the chamber is UV transmissive. The term “transmissive” means that the UV light from the UV source 432 is transmitted to and penetrates through the chamber 430. For example, the chamber 430 may be transparent with the ability to allow UV-C to penetrate so that photocatalytic oxidation takes place within the fluid transport chamber 430.

In some embodiments, the fluid transport chamber 430 may have a coil shape and hereinafter sometimes referred to as “the UV transmissive coil 430,” within which photocatalytic oxidation takes place. The UV transmissive coil 430 is non-plugged and is fluidly coupled to the liquid connector 163 connected to the cover 160 to receive the contaminated liquid from the influent line. The effluent to the coil 430 may be coupled to receive, be mixed with, or dosed with an amount of hydrogen peroxide (H₂O₂) or other AOP additive. An example UV light is described in U.S. Pat. No. 10,046,075 incorporated herein by reference.

When the liquid is first pumped, via the influent pump 135 (FIG. 1), into the system 100, the liquid is dosed with a known concentration of hydrogen peroxide (H₂O₂) or other AOP additive. The dosed liquid is then fed into one or more of the onboard reactors 150 having one or more UV interior chamber reactors 400.

In some embodiments, the H₂O₂ or AOP liquid dosing is actually at the influent line (i.e., fluid pipes 170) outside and on the back of the housing for the mixture to enter the UV reactors. This may include an inline mixer 177 to mix the mixture.

In some embodiments, the dosed liquid may be fed in parallel to the two (or more) reactors 150. In some embodiments, the dosed liquid may be fed in series using a metering valve configuration.

As shown in FIG. 1, dosing of H₂O₂ or AOP may be dosed/pumped via a metering valve MV1 based on concentration; parts per million (PPM). The dosing is based on the inner diameter of the chamber specified for the metering valve and the speed at which the metering valve is turning. This is all tuned with the system flow, in gallons per minute, to arrive the concentrated value of PPM.

The UV sources (i.e., UV transmissive tubes 432) are encapsulated in a UV transmissive lens material wrap. This protects the bulb from any type of fouling. It also acts as a safety barrier in the case the bulb is broken. The wrap will keep the mercury from the lamp from contaminating the contaminated liquid such as groundwater. UV sources may include UV lamps and UV light emitting diodes (LEDs). In some embodiments, the UV sources may include UV LEDs (not shown) affixed to the interior walls of within the reactor chamber 152. A UV source may include one UV LED or multiple UV LEDs. The UV LEDs may be arranged in a column to form an elongated UV lamp and emit light in 360 degrees. Thus, these UV LEDs may be used in any reactor chamber described herein to provide a double dose of the UV light.

In some embodiments, in addition to the UV reactor design (non-plug flow), a quartz sleeve is not used because the inventors have determined that the quartz sleeve degrades over time when exposed to UV. After it degrades/etched, it is susceptible to biofouling which leads to a degradation of UV dosing performance since now the UV light from the UV source cannot get passed the quartz sleeve material and effectively dose the PCB molecule on the outside of the quartz sleeve.

Additionally, the inventors have determined in prior art systems space of air between the UV source and the inside of the quartz sleeve becomes another medium for UV loss. Lastly, since the quartz sleeve prevents the cool water from being directly in contact with the UV source, the UV sources have a shorter lifetime due to thermal stress. The embodiments herein solve these problems. The UV interior chamber reactors, reactor chambers and/or reactors may be quartz-sleeveless reactors such that a quartz-sleeve is not used by the reactor, within the chamber or part of any of the UV interior chamber reactor.

The treated mixture of each interior chamber reactor enters the reactor chamber where the reactor chamber can become filled with an amount of the treated mixture. The treated mixture also simultaneously surrounds the UV sources to cool the UV sources as the UV sources further process untreated mixture within the chamber 430. Furthermore, the treated mixture can be further treated in the reactor chamber by any of the UV sources of any of the interior chamber reactors in the reactor chamber.

The UV transmissive coil 430 allows for a “double dosing” effect of UV-C. As the liquid flows through the UV transmissive coil 430, the liquid is kept close to the UV source 432. This allows the liquid to receive the highest dosing rate of UV-C. The UV transmissive coil 430 then discharges from a continuous-batch flow discharge port 435 into the bottom of the reactor chamber 152 (FIG. 5) causing turbidity inside the reactor 150. Although there are four interior chamber reactors found in each reactor chamber 152, increase in dimensions of the chamber 152 may provide for additional interior chamber reactors. The liquid eventually discharges the reactor chamber 152 via the effluent line 159 or pipe 509 that is coupled to the first gauge 154. This completes the second part of the “double dosing” effect. Since this is not a plug flow design, where the UV coil exits the reactor, instead, the coil 430 exits inside the reactor chamber 152 allowing for the PCB molecule to further mix and be more exposed to more UV and H₂O₂ hydroxyl radical exposure while in the reactor 150 prior to exiting the effluent line 159 (FIG. 1). In the embodiment of FIG. 4, the UV coil 430 provides two coiled chamber members, which wrap around the center UV source 432. The two chamber members are helically wound around the center tube of the UV source. In some embodiments, the chamber is a single tube that is helically wound around the center tube of the UV source.

The reactor chamber 152 may be a quartz-sleeveless reactor chamber 152 such that it does not include a quartz sleeve. The UV interior chamber reactor 400 does not include a quartz sleeve and is sometimes referred to as a quartz-sleeveless UV interior chamber reactor.

In operation, UV light sensor 505 senses the light of each UV source 432. If any lamp goes out, the UV light sensor 505 provides a representative signal of a sensed UV source condition, for example, to control station 190. In other embodiments, the sensed UV source condition may include OFF or ON. Further, the sensor 505 may determine that the UV source is illuminating below or near a certain threshold. The representative signal may cause a technician to change the interior chamber reactor 400 or UV source.

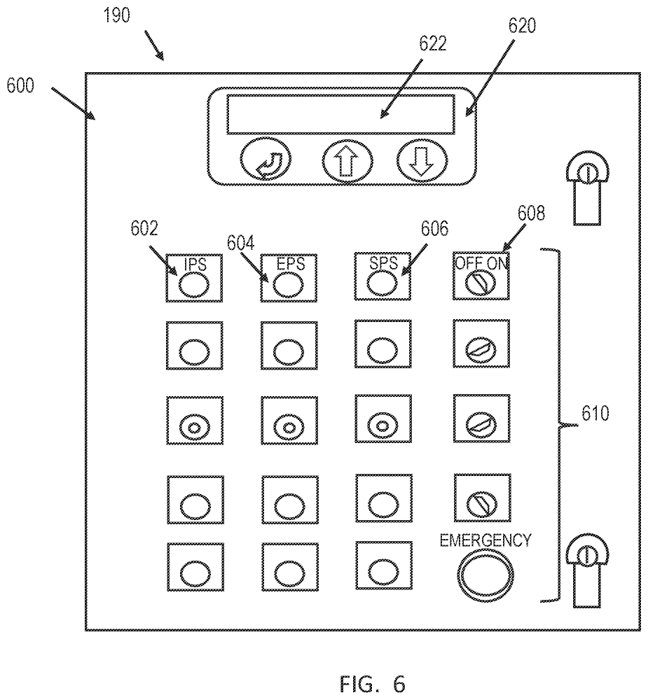

FIG. 6 shows a front view of the control panel 600 according to an embodiment. The control station 190 includes the control panel 600. The control panel 600 may include a plurality of control buttons and switches 610. Some of the control buttons include visual indicators such as an LED indicator. The control station 190 may also include a sensor display panel 620 configured to display on display device 622 various sensor readings or notifications. In some embodiments, the control station 190 may also communicate remotely using wired or wireless communication systems. The controller 191 may control the UV/AOP process where the UV/AOP may be performed autonomously.

In some embodiments, the system 100 can be run in either a series or parallel configuration. This allows for either a higher UV dosing or a higher flowrate. This can be determined by the operator via the control station 190. The flowrates are also able to be controlled by the operator.

In some embodiments, the control buttons and switches may include illuminated buttons. The buttons, for example, turn on various pumps. When the pump is on, the button may illuminate a first color indicative of ON such as, without limitation, green. When the device is OFF, the button may illuminate a second color indicative of OFF. The button 602 may start the influent pump 135 (FIG. 1) where influent pump start (IPS) button may be labeled. The button 604 may start the effluent pump 179 (FIG. 1) where effluent pump start (EPS) button may be labeled. The button 606 may start a series pump 195 (FIG. 1) where series pump start (SPS) button may be labeled. The switch 608 may provide for turning ON or OFF of the maintenance light. The switch 608 may be a manual switch.

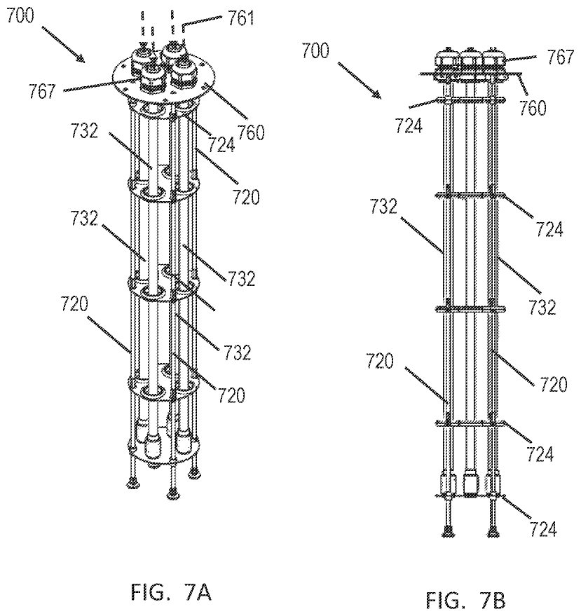

The one or more illuminating devices 120 (FIG. 2B) of system 100 are configured to show a status state via internally mounted LED lights through the illuminating panels 117 (FIG. 1). These lights are visible from outside the housing 103 due to the illuminating panels 117 being frosted acrylic, for example. Other material may be used for the panels, provided the panel material has a degree of transparency to allow the panel to allow light to shine through, in some embodiments. The control panel 600 and control station 190 may be electrically connected to components of the housing, the one or more reactors 150, the light source 120, and the at least one interior chamber reactor 400 or 700 (FIGS. 7A-7B). The control panel 600 and control station 190 may be electrically coupled to the UV ballast 306 to provide power to the UV ballast or other power source. The control panel 600 and control station 190 may be electrically and communicatively coupled to other electrically powered or controlled components and/or sensors to automate the process.

The system 100 may use a plurality of different colors that all represent different functioning states of the system 100. Examples of varying colors are described below.

Pulsing Blue: This state may signify that the system 100 is in an idle state. The only way the system 100 can come out of this state is if all pumps are running and the UV sources are all on.

Solid Amber: This state signifies that the system 100 has been set to have all eight UV sources turned to “On,” but one or more of the lamps is not functioning. This can be due to a failed UV Ballast or Lamp.

Solid Green: This state signifies that the system 100 is fully operational. All the pumps and UV sources are “On.”

Solid White: This state signifies that the maintenance light has been set to “On.” This allows the operator to perform maintenance or other work inside the housing.

Solid Red: This state signifies that one or more of the emergency stops have been triggered. In this state, none of the normal operations will be allowed to commence.

Unless otherwise defined, all terms (including technical and scientific terms) used herein have the same meaning as commonly understood by one of ordinary skill in the art to which embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The above example colors are examples and the colors should not be limited to these colors in any way.

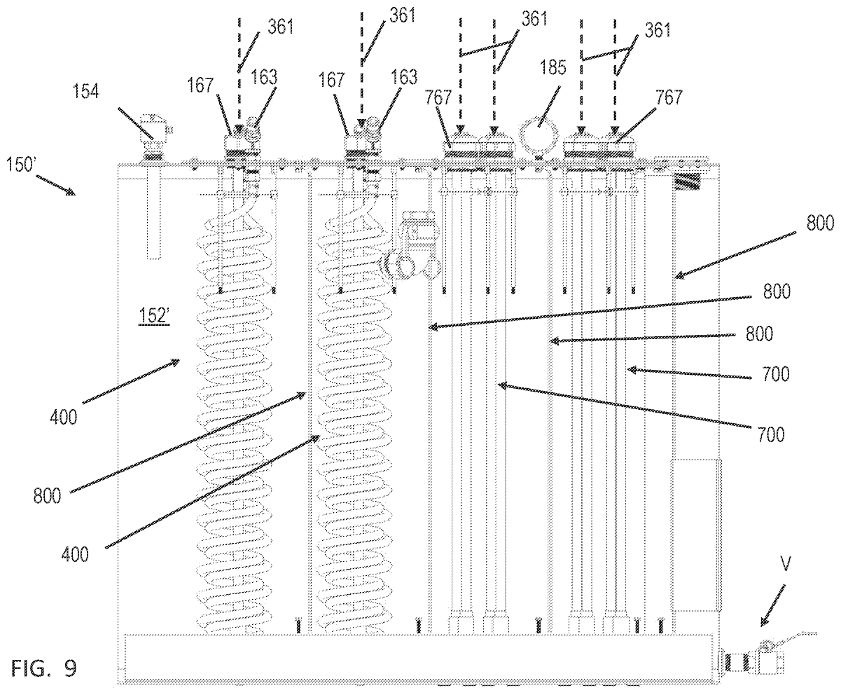

FIGS. 7A and 7B show perspective and front views of a second ultraviolet (UV) interior chamber reactor 700. FIG. 9 shows an interior view of the reactor 150′ according to an embodiment for use in system 100 (FIG. 1). The second UV interior chamber reactor 700 is similar UV interior chamber reactor 400, but does not include a fluid transport line (i.e., coil 430). Instead, the second UV interior chamber reactor 700 provides additional sources of UV light to continue radiating the treated fluid in the reactor chamber that was discharged from the UV interior chamber reactor 400. The second UV interior chamber reactor 700 is also quartz-sleeveless.

The second UV interior chamber reactor 700 includes a plurality of ultraviolet (UV) lamps 732 that are parallel to each other. The lamps 732 may be equidistant from each other with spacing to allow liquid to flow therebetween. The UV sources 732 may be similar to the UV sources 432 previously described. For example, the UV sources 732 may be encapsulated in a UV transmissive lens material wrap.

The second UV interior chamber reactor 700 includes a cover 760 configured to be removably coupled to the top of the reactor 150′. The reactor chamber 152′ has a plurality of orifices in the top. These orifices are dimensioned to be closed or sealed by cover 760 and receive remaining portion of the interior chamber reactor 700. The second UV interior chamber reactor 700 includes at least one electrical connector 767 to supply power to the UV sources 732 via flexible chamber/cable 761 to transfer the power to the UV interior chamber reactor 700. The power may be supplied from a UV ballast 306 (FIG. 3B). The second UV interior chamber reactor 700 may include a plurality of support rods 720 coupled to one or more substrates 724. The UV source is supported by the substrates 724. For example, the UV sources may be positioned at 45°, 135°, 225° and 315°. The substrates 724 have a diameter which is smaller than the diameter of the cover 760 so that the substrates can be received through the orifices in the reactor. The UV interior chamber reactor 700 includes fasteners to fasten the cover 760 to the reactor chamber 152′.

Referring again to FIG. 9, the reactor 150′ may include one or more first UV interior chamber reactors 400 and one or more second UV interior chamber reactors 700. Each first interior chamber reactor and each second interior chamber reactor comprise an interior chamber reactor cover configured to be removably coupled to the reactor chamber. In some embodiments, the one or more second UV interior chamber reactors 700 are positioned parallel to and downstream the one or more first UV interior chamber reactors 400 within the reactor chamber. To prevent overcrowding in FIG. 9, portions of the rods 420 and 720 have been removed. The second UV interior chamber reactors 700 may be quartz-sleeveless interior chamber reactors which do not include a quartz sleeve.

In the embodiment of FIG. 9, two first UV interior chamber reactors 400 are installed in series followed by two second UV interior chamber reactors 700. However, the UV interior chamber reactors may be alternated, such that the UV interior chamber reactors may include a first UV interior chamber reactor 400 followed by a second UV interior chamber reactor 700, followed by another first UV interior chamber reactor 400 and another second UV interior chamber reactor 700. The addition of at least one second UV interior chamber reactor 700 provides the benefit of additional UV energy sources in the fluid path of the treated liquid to increase the rate of destruction of contaminants within the reaction chamber 152′. In FIG. 9, the double dosing may be accomplished by subjecting the discharged treated mixture from either of UV interior chamber reactors 400 to further UV light by the UV sources of UV interior chamber reactors 400 and/or UV interior chamber reactors 700.

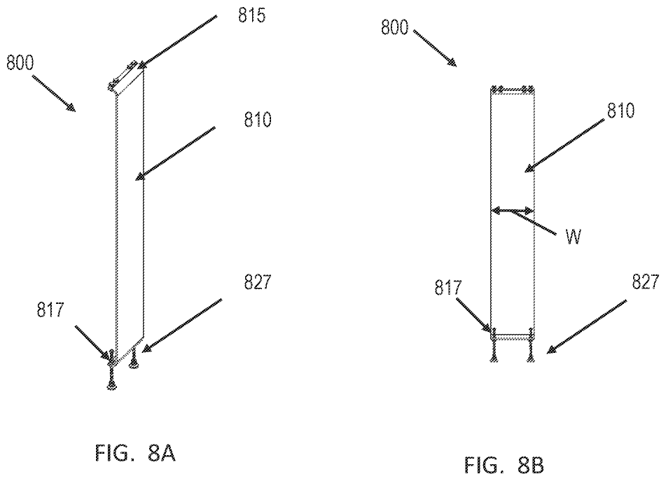

FIGS. 8A and 8B show side perspective and front views of a baffle 800 according to an embodiment. The baffles 800 may comprise an elongated structure 810 that has a thin profile. The elongated structure 810 may be sheet metal. The structure 810 includes first connectors 815 and second connectors 817 to fasten or bolt the structure 810 to wall surfaces within the reactor chamber 152. The baffles 800 are placed within the liquid stream to block or disrupt flow of the treated liquid. The baffles 800 may include support feet 827. The baffle 800 causes flow to be turbulent to break up streams of the fluid passing lights too quickly. The at least one baffle mounted in the reactor chamber may cause turbulence of the radiated resultant mixture and retard flow of the radiated resultant mixture to the outlet port.

Returning again to FIG. 9, the baffles 800 are shown fastened within the interior reactor chamber 152′. The baffles 800 may be positioned between adjacent UV interior chamber reactors 400 and/or UV interior chamber reactors 700. A baffle 800 may be positioned after the last UV interior chamber reactor, such as UV interior chamber reactor 700. Each baffle 800 is angled or perpendicular to a longitudinal length of the reactor chamber 152′. The width W of the structure 810 is smaller than the inner width of the reaction chamber.

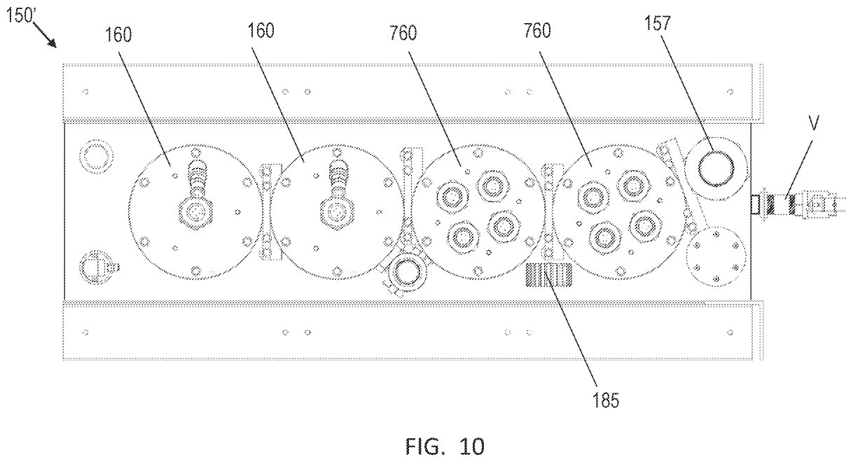

FIG. 10 shows a top view of the reactor 150′ of FIG. 9 with covers 160 for the UV interior chamber reactors 400 and the covers 760 for UV interior chamber reactors 700 installed.

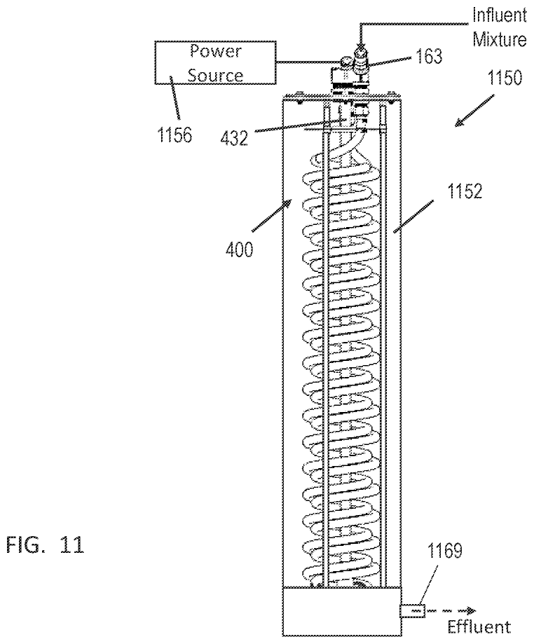

FIG. 11 shows an interior view of a tubular reactor 1150 according to an embodiment. The tubular reactor 1150 includes a reactor chamber 1152 that houses a single UV interior chamber reactor 400, for example. The tubular reactor 1150 may be a quartz-sleeveless reactor 1150. The reactor 1150 may receive power from power source 1156. To power the UV source 432. The reactor 1150 includes a liquid connector 163 to receive a mixture of the contaminated liquid and an AOP additive, such as hydrogen peroxide. The reactor chamber 1152 may include a discharge port 1169.

The reactor 1150 may treat an amount of contaminated media of liquid in the field for site testing and treating. The reactor 1150 may be used to perform lab studies on sites in the field.

In some embodiments, the tubular reactor 1150 may receive a UV interior chamber reactor 700. In this configuration, an amount of the mixture (including contaminated liquid and an AOP additive) would be placed within the chamber 1152. Then the UV interior chamber reactor 700 can be installed within the chamber 1152. In operation, the UV sources are powered for a set amount of time to radiate the mixture before discharging the radiated mixture to decontaminate the contaminated liquid. In some embodiments, the UV interior chamber reactors 400 and 700 may be interchangeable from within the reactor chamber 1152 for a two-step process.

The reactor 1150 provides a “double dosing” effect to treat the mixture. In the embodiment using UV interior chamber reactor 400, the mixture receives a dose of UV radiation in the chamber 430. The discharged (treated) mixture exiting the chamber 430 will also receive another dose of UV radiation.

The reactor 1150 may provide a second dose of UV radiation using the interchangeable UV interior chamber reactor 700 to further treat the discharged (treated) mixture remaining in the chamber 1152.

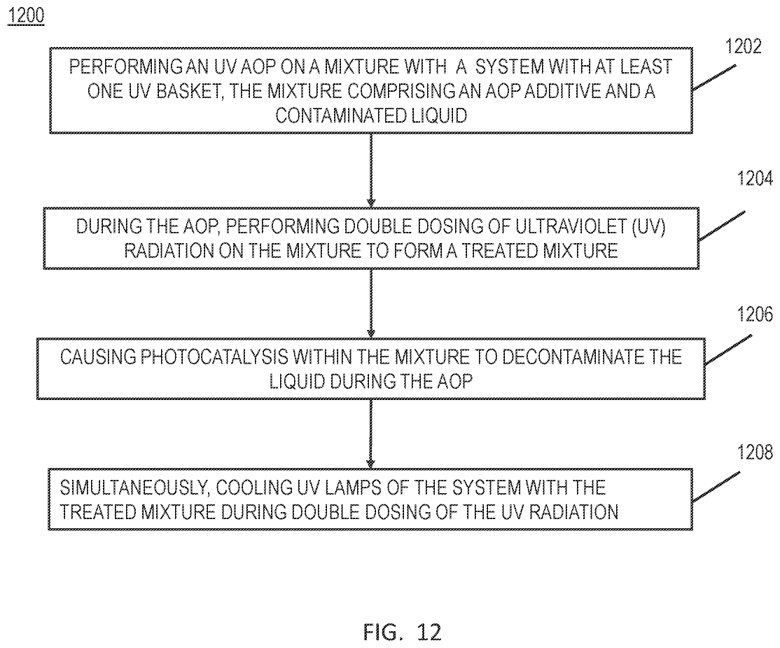

FIG. 12 shows a flow diagram of a method 1200 for decontaminating a contaminated liquid. The method 1200 may include, at block 1202, performing an ultraviolet advanced oxidation process (AOP) on a mixture with a system 100 having at least one interior chamber reactor, such as UV interior chamber reactor 400. The mixture includes an AOP additive and a contaminated liquid. The method 1200 may include, at block 1204, during the AOP, performing double dosing of ultraviolet (UV) radiation on the mixture to form a treated mixture. The method 1200 may include, at block 1206, causing photocatalysis within the mixture to decontaminate the liquid, during the AOP. The UV radiation includes UV-C wavelengths. The method may include, at block 1208, simultaneously, cooling UV sources of the system 100 with the treated mixture during double dosing of the UV radiation.

The method 1200 can be used to decontaminate a contaminated liquid that includes one of: Ethanol, transformer oil, mineral oil, water, groundwater, reclaimed liquid, and a contaminated fluid. The AOP additive includes one of: hydrogen peroxide, titanium dioxide and a chemical composition that causes photocatalysis within the mixture to decontaminate the liquid using UV radiation or light.

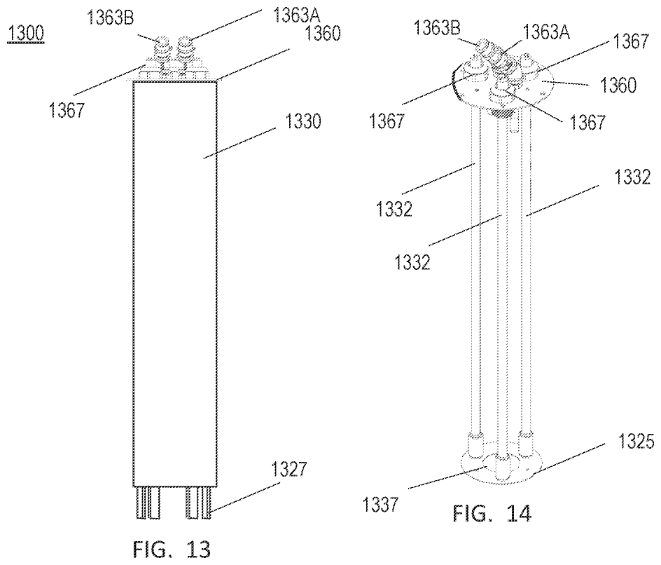

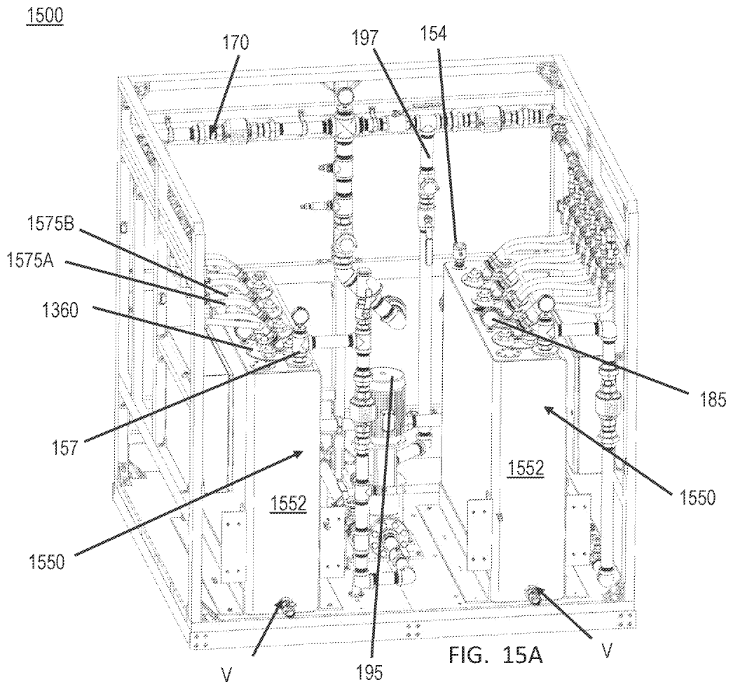

FIG. 13 shows a front view of the third ultraviolet (UV) interior chamber reactor 1300. FIG. 14 shows the third ultraviolet (UV) interior chamber reactor 1300 with the outer chamber removed. The continuous-batch flow, UV interior chamber reactor 1300 may include a plurality of UV transmissive tubes 1332 which are UV sources 1332. The UV interior chamber reactor 1300 may include non-transmissive fluid transport chamber 1330 which encloses (houses and surrounds) all of the UV transmissive tubes 1332. The UV interior chamber reactor 1300 include feet 1327 at an end of the fluid transport chamber 1330 closest to the bottom of the reactor chamber.

The non-transmissive fluid transport chamber 1330 receives a mixture of the contaminated liquid and the AOP additive via liquid connectors 1363A and 1363B connected to the cover 1360. The non-transmissive fluid transport chamber 1330 is where photocatalytic oxidation takes place. The effluent to the non-transmissive fluid transport chamber 430 may be coupled to receive, be mixed with, or dosed with an amount of hydrogen peroxide (H₂O₂) or other AOP additive. The non-transmissive fluid transport chamber 1330 may be made of aluminum or other material. The material being selected to be non-corrosive in the presence of the mixture or to limit fouling. The non-transmissive fluid transport chamber 1330 allows the treated mixture of each interior chamber reactor to enter the reactor chamber where the reactor chamber can become filled with an amount of the treated mixture. The treated mixture in the interior chamber reactor 1300 also simultaneously surrounds the UV sources to cool the UV sources as the UV sources further process untreated mixture within the chamber 1330.

The cover 1360 of the UV interior chamber reactor 1300 includes a plurality of electrical connectors 1367. Each electrical connector 1367 is connected to the UV source 1332. As described previously, electrical cables connect to a UV ballast to provide power to the UV sources to produce UV radiation that includes UV-C wavelengths.

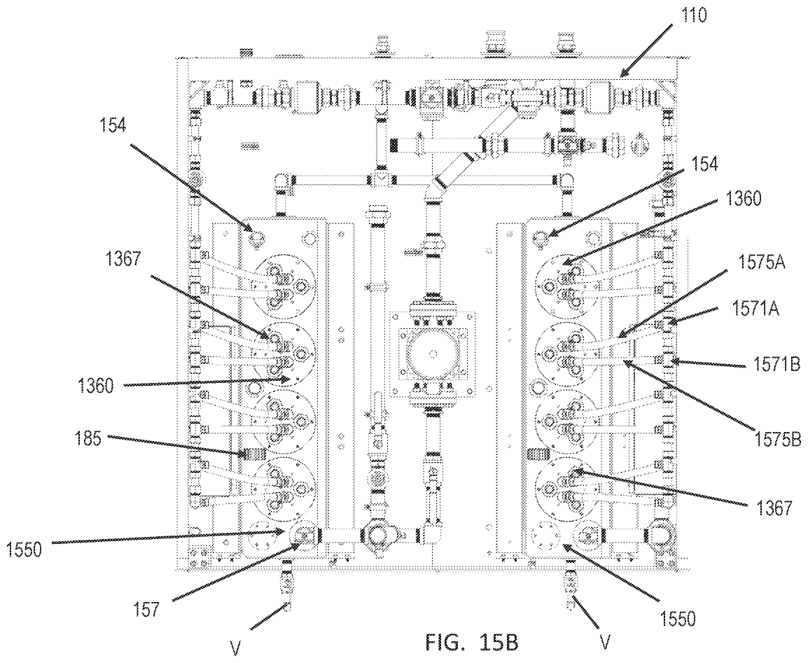

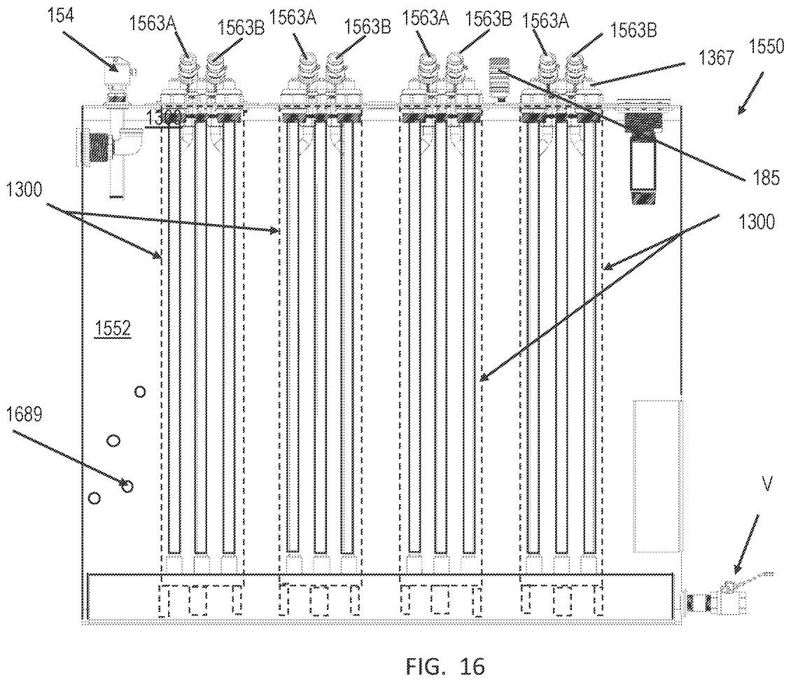

The UV interior chamber reactor 1300 may include a bottom substrate 1325 which has an unplugged orifice 1337. This allows the mixture in the non-transmissive fluid transport chamber 1330 to exit into the reactor chamber 1552 (FIGS. 15A, 15B and 16).

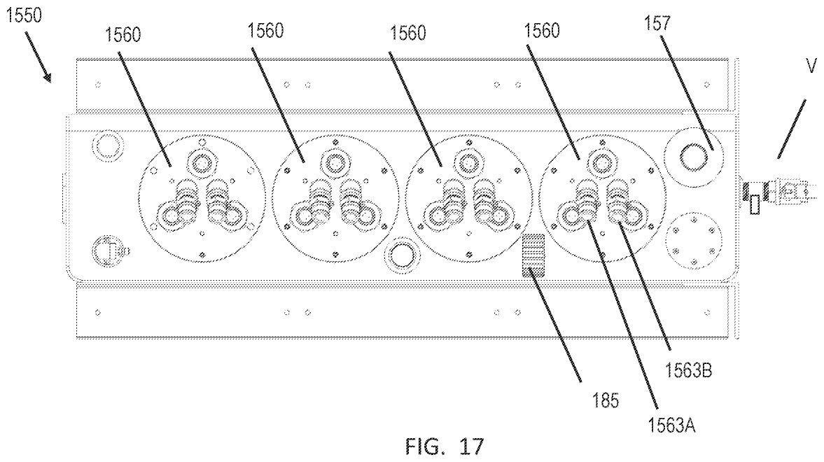

Referring also to FIGS. 15A and 15B, the system 1500 is similar to the system 100 (FIGS. 2A, 2B, 3A and 3B) described above. Thus, only the differences will be described below. FIG. 16 shows an interior view of the reactor according to an embodiment with UV LEDs 1689. To prevent overcrowding of the drawing, only a few UV LEDs 1689 are shown. However, the UV LEDs 1689 may be placed in a matrix of rows and columns to provide a second dosing (double dosing) of the liquid. FIG. 17 shows a top view of the reactor of FIG. 16.

The system 1500 includes one or more reactors 1550. In the illustration, two reactors 1550 are shown which may be quartz-sleeveless. However, the system 1500 may be sized to accommodate an amount of liquid that needs decontamination. Each reactor 1550 includes a reactor chamber 1552 with a plurality of orifices for loading the ultraviolet (UV) interior chamber reactor 1300 (FIG. 13); only the interior chamber reactor cover 1360 is shown. The reactor 1550 may have a first gauge 154 connected to the reactor chamber 1552. The first gauge 154 measures effluent pipping pressure. The reactor 1550 may have a second gauge 185 connected to the reactor chamber 1552. The reactor chamber 1552 includes chamber walls that form an enclosure with orifices and other ports or outlets. The second gauge 185 measures the reactor pressure. The reactor chamber 1552 may include various ports with valves V connected thereto. For the sake of brevity, only some of the values and ports are labeled. The reactor 1550 may include a UV light sensor (not shown). For example, each interior chamber reactor 1300 may include a UV light sensor within the chamber 1330 to determine when a UV source is dim or non-functional.

The system 1500 may include fluid pipes 170 which allow certain liquids to flow to the liquid interfaces 1575A and 1575B, each liquid interface being connected to cover 1360 of the UV interior chamber reactors (FIG. 4) via liquid connectors 1563A and 1563B, respectively. Fluid pipes 170 may be influent lines that are connected to an influent pump 135 (FIG. 1). For example, pipes 170 may transport the contaminated liquid, such as liquid to the reactor 1550. The fluid pipes 170 may include pipe branches 1571A and 1571B. Each pipe branch 1571A and 1571B is connected to a liquid interface 1575A and 1575B, respectively. The liquid interfaces may include flexible chamber.

The treated mixture from the first reactor may leave the reactor chamber through the outlet line 157 to series pump 195 and through a return line 197. Then the treated mixture in the return line 197 may be further processed in a second reactor chamber.

In some embodiments, the reactor 1550 may include one or more UV interior chamber reactors 1300 and one or more UV interior chamber reactors 700. The UV interior chamber reactors 700 may provide UV dosing of the treated mixture from UV interior chamber reactor 1300 within the reactor chamber.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order or importance, but rather the terms first, second, etc., are used to distinguish one element from another. As used herein the expression “at least one of A and B,” will be understood to mean only A, only B, or both A and B.

While various disclosed embodiments have been described above, it should be understood that they have been presented by way of example only, and not limitation. Numerous changes, omissions and/or additions to the subject matter disclosed herein can be made in accordance with the embodiments disclosed herein without departing from the spirit or scope of the embodiments. Also, equivalents may be substituted for elements thereof without departing from the spirit and scope of the embodiments. In addition, while a particular feature may have been disclosed with respect to only one of several implementations, such feature may be combined with one or more other features of the other implementations as may be desired and advantageous for any given or particular application. Furthermore, many modifications may be made to adapt a particular situation or material to the teachings of the embodiments without departing from the scope thereof.

Further, the purpose of the foregoing Abstract is to enable the U.S. Patent and Trademark Office and the public generally and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of the present disclosure in any way.

Therefore, the breadth and scope of the subject matter provided herein should not be limited by any of the above explicitly described embodiments. Rather, the scope of the embodiments should be defined in accordance with the following claims and their equivalents.

What is claimed is:

1. A system comprising:
one or more quartz-sleeveless reactors, each quartz-sleeveless reactor including an independent and continuous interior reactor chamber and at least one continuous-batch flow, interior chamber reactor housed in the interior reactor chamber, each interior chamber reactor includes at least one ultraviolet (UV) lamp to emit UV radiation and fluid transport chamber, each interior chamber reactor is configured to:
pass a stream of a mixture in the fluid transport chamber and around the at least one UV lamp, the mixture comprising an advanced oxidative process (AOP) additive and contaminated liquid;
radiate the mixture while in the fluid transport chamber with the emitted UV radiation from the at least one UV lamp;
simultaneously cool the at least one UV lamp with the mixture; and
autonomously pass a radiated resultant mixture into the interior reactor chamber.

2. The system of claim 1, wherein:
the at least one continuous-batch flow, interior chamber reactor comprises a plurality of interior chamber reactors; and
the radiated resultant mixture continues to receive the UV radiation produced by the at least one UV lamp of any interior chamber reactor the plurality of interior chamber reactor to further treat the radiated resultant mixture.

3. The system according to claim 1, wherein:
the at least one continuous-batch flow, interior chamber reactor includes at least one first interior chamber reactor, and each quartz-sleeveless reactor further comprising:
at least one second interior chamber reactor, each second interior chamber reactor comprises a plurality of ultraviolet (UV) lamps that are parallel to each other and a second interior chamber reactor cover configured to be removably coupled to the reactor chamber,
wherein the second interior chamber reactor is positioned parallel to and downstream the at least one first interior chamber reactor.

4. The system according to claim 3, wherein the reactor chamber comprises:
an outlet port at one end of the reactor chamber; and
at least one baffle mounted in the reactor chamber to cause turbulence of the radiated resultant mixture and retard flow of the radiated resultant mixture to the outlet port,
a width of the at least one baffle is angled or perpendicular to a longitudinal length of the reactor chamber.

5. The system according to claim 1, wherein the UV radiation is ultraviolet light C having a wavelength in the range of 100-280 nanometers (nm).

6. The system according to claim 1, wherein the fluid transport chamber includes two coiled chamber members made of UV transmissive lens material, the fluid transport chamber is helically wound around and along a length of the UV lamp.

7. The system according to claim 1, wherein the contaminated liquid includes one of: Ethanol, transformer oil, mineral oil, water, groundwater, reclaimed liquid, and a contaminated fluid.

8. The system according to claim 1, further comprising:
an influent liquid input to receive the contaminated liquid;
a supply tank to store the advanced oxidative process (AOP) additive; and
a metered valve coupled to the supply tank and to the influent liquid input to dose the contaminated liquid with a metered amount of the AOP additive.

9. The system according to claim 1, wherein the AOP additive comprises one of hydrogen peroxide (H₂O₂) or titanium dioxide (TiO₂).

10. The system according to claim 1, further comprising:
a light source to emit a plurality of different colors which represent different functioning states of the system; and
a housing having an interior to house the one or more reactors and the light source, the housing includes at least one panel to illuminate light of the light source from the interior to the exterior.

11. The system according to claim 10, further comprising a control panel coupled to the housing, the one or more reactors, the light source, and the at least one interior chamber reactor.

12. The system according to claim 1, wherein the fluid transport chamber comprises one of UV transmissive material and UV non-transmissive material.

13. The system according to claim 12, wherein the fluid transport chamber is unplugged, made of UV non-transmissive material, and houses and surrounds the at least one UV source.

14. An interior chamber reactor comprising:
a cover having an electrical connector to receive power from an ultraviolet ballast and a fluid connector;
an ultraviolet (UV) lamp to emit UV radiation and electrically connected to the electrical connector in the cover; and
a UV transmissive fluid transport chamber made of UV transmissive lens material, the fluid transport chamber to:
pass a stream of a mixture through the fluid transport chamber coiled around the UV lamp, the mixture comprising an advanced oxidative process (AOP) additive and contaminated liquid,
pass the UV radiation by the UV lamp along a length of the chamber to radiate the mixture while in the chamber, and
pass a radiated resultant mixture into a reactor chamber such that the radiated resultant mixture continues to receive the UV radiation produced by the UV lamp to further treat the radiated resultant mixture, and simultaneously cool the UV lamp by the radiated resultant mixture.

15. The interior chamber reactor according to claim 14, wherein the UV radiation is ultraviolet light C having a wavelength in the range of 100-280 nanometers (nm).

16. The interior chamber reactor according to claim 14, wherein the UV transmissive fluid transport chamber includes two coiled chamber members made of UV transmissive lens material, the fluid transport chamber is helically wound around and along a length of the UV lamp.

17. The interior chamber reactor according to claim 16, further comprising:
a plurality of substrates having a diameter; and
a plurality of support rods coupled to the substrates,
wherein the UV lamp is supported in a center of the plurality of substrates.

18. The interior chamber reactor according to claim 16, wherein the UV transmissive fluid transport chamber is helically wound around and along a length of the UV lamp.

19. The interior chamber reactor according to claim 14, wherein the UV lamp and the UV transmissive fluid transport chamber together perform an ultraviolet advanced oxidation process with double dosing of the mixture with the UV radiation.

20. A method comprising:
performing an ultraviolet advanced oxidation process (AOP) on a mixture with the system of claim 1, the mixture comprising an AOP additive and a contaminated liquid;
during the AOP, performing double dosing of ultraviolet (UV) radiation on the mixture to form a treated mixture;
causing photocatalysis within the mixture to decontaminate the liquid during the AOP;
and
simultaneously, cooling UV lamps of the system with the treated mixture during double dosing of the UV radiation.

21. The method according to claim 20, wherein:
the at least one interior chamber reactor is a first interior chamber reactor in a reactor chamber; and
the performing of the ultraviolet AOP on the mixture further comprises:
performing the ultraviolet AOP in the reactor chamber using a second interior chamber reactor different from the first interior chamber reactor, at a location downstream the first interior chamber reactor in the reactor chamber, the second interior chamber reactor comprising a plurality of parallel UV lamps to emit UV radiation.

22. The method according to claim 21, wherein the UV radiation is ultraviolet light C having a wavelength in the range of 100-280 nanometers (nm).

23. The method according to claim 22, wherein:
the contaminated liquid includes one of: Ethanol, transformer oil, mineral oil, water, groundwater, reclaimed liquid, and a contaminated fluid; and
the AOP additive includes one of: hydrogen peroxide and titanium dioxide.
